(19) United States
(12) Patent Application Publication
(10) Pub. No.: US 2026/0272329 A1
(43) Pub. Date: Sep. 17, 2026

(54) SYSTEMS AND METHODS FOR MONITORING BODY ORIENTATION OF A SUBJECT

(71) Applicant: Growth Armor, LLC, Wilmington, DE (US)

(72) Inventor: Michael A. BAIN, Costa Mesa, CA (US)

(21) Appl. No.: 19/673,586

(22) Filed: May 11, 2026

Related U.S. Application Data
(63) Continuation-in-part of application No. 18/802,889, filed on Aug. 13, 2024, now Pat. No. 12,667,277.

(51) Int. Cl. A61B 5/11

(52) U.S. Cl. CPC A61B 5/1113; A61B 2560/0266

(57) ABSTRACT

Systems and methods are disclosed for determining a body orientation of a subject. A device may be secured to the subject which comprises a housing, a first pair of electrodes in an internal chamber defined by the device, and a conductive material in the internal chamber. The first pair of electrodes may be associated with a first orientation of the device such that when the device is placed in the first orientation, the conductive material is positioned, due to the effects of gravity, to complete a first circuit between the first pair of electrodes. Further pairs of electrodes may be included, each of which may have their circuits completed by the conductive material when the device is placed in a certain orientation. Readings from these pairs of electrodes may be used to determine the subject’s body orientation.

### CROSS-REFERENCE TO RELATED APPLICATIONS

This application is a continuation-in-part of Application No. 18/802,889, filed August 13, 2024, the entire contents of which are incorporated herein by reference.

### STATEMENT RE: FEDERALLY SPONSORED RESEARCH/DEVELOPMENT

Not Applicable

### BACKGROUND

### 1. Technical field

The present application relates to monitoring a body orientation of a subject. More specifically, the present application relates to devices containing a conductive material and pairs of electrodes which are securable to a subject such that the conductive material completes a circuit between a particular pair of electrodes, depending on a body orientation of the subject. The present application also relates to devices configured to monitor body orientation, monitor repositioning compliance, alert caregivers, facilitate institutional documentation, and monitor system integrity.

### 2. Related Art

Pressure injuries, which also go by the names of pressure ulcers or bed sores, occur when pressure is applied to the skin over a long period of time. These injuries are most prevalent for patients recovering from medical conditions that leave them bedridden, with most cases happening on the heels of the feet, shoulder blades, back of the head, and tailbone area. An easy way to prevent these injuries from arising is to turn a patient and change their body orientation every once in a while, with most hospitals using a standard of doing so once every two hours.

It can, however, be just as easy for a hospital technician to forget to do this. The use of timed alarms has not proven to be a surefire reminder to these technicians, as they have several duties which can distract them from this duty. Smith & Nephew PLC, headquartered in England, UK, provides a product called the LEAF Patient Monitoring System for monitoring a patient’s body orientation and notifying nearby staff when the patient needs to be turned. However, this device measures the patient’s movement with an accelerometer, which consumes a relatively large amount of energy, which drives up the cost of the device and drains the battery quite quickly. As such, it can be seen that new and improved systems and methods for monitoring the body orientation of a subject are needed and desired in the art which can be used for long periods of time and are cheaper to manufacture.

Patient body-orientation monitoring systems can be used in care environments in which a patient is periodically repositioned to reduce localized pressure and to support pressure-injury prevention protocols. In such environments, however, detecting that a patient has changed position does not necessarily provide a reliable institutional record showing whether a prescribed repositioning interval was satisfied, whether a detected movement constituted a clinically meaningful offloading event, or whether a missed-turn event occurred. Many facilities also differ in their available documentation infrastructure. For example, some facilities may use electronic health record systems or institutional servers, while other facilities may rely on local computers, bedside hubs, printers, local memory, or later-downloaded records. Conventional manual charting may be retrospective and may not identify, at the time of occurrence, the specific type of repositioning event, the timing of the event, the source of the event data, or whether the event was stored, transmitted, printed, acknowledged, or escalated.

Body-orientation monitoring systems may also be used in infant-care settings to notify a caregiver when an infant assumes an undesired body orientation. In such settings, the absence of a position alert can be misinterpreted as an indication that the infant remains in a desired position, even though the monitoring apparatus may be unable to detect or communicate an orientation condition. For example, a sensor pod may be disconnected, out of communication range, low on battery, removed from or misaligned relative to a garment or support structure, malfunctioning, or unable to cause a caregiver device to generate a notification. Accordingly, a body-orientation monitoring system that only alerts when a particular body orientation is detected may not adequately inform a caregiver when monitoring capability itself has become unavailable or unreliable.

### BRIEF SUMMARY

To solve these and other problems, the present disclosure provides systems and methods for monitoring a subject’s body orientation. A device for monitoring a body orientation of a subject the device may comprise a housing, a first pair of electrodes in an internal chamber defined by the device, the first pair of electrodes being associated with a first orientation of the device, and a conductive material in the internal chamber, the conductive material completing a first circuit between the first pair of electrodes when the device is placed in the first orientation. In particular, gravity may cause the conductive material to move through the internal chamber and be placed in contact with the pair of electrodes when the device is placed in this first orientation. This device could further comprise a second pair of electrodes in the internal chamber, the second pair of electrodes being associated with a second orientation of the device, the conductive material completing a second circuit between the second pair of electrodes when the device is placed in the second orientation. To measure further orientations, a third, fourth, fifth, etc. pair of electrodes can be included for a third, fourth, fifth, etc. orientation of the device. By securing this device to a subject and correlating the device’s orientation with certain body orientations of that subject, one can monitor the body orientation of the subject based on the electrode readings.

If multiple pairs of electrodes are present, they may be spaced from one another such that a rotation of the device on an axis of rotation by at least 30 degrees, at least 60 degrees, or at least 90 degrees is necessary to transition the device from one orientation to a second orientation. The pairs of electrodes can be disposed on an internal surface which bounds the internal chamber. This internal surface can have a circular geometry.

The device may further comprise a battery and an alarm. The alarm could be operative to sound in response to one or more of: the first circuit being completed for at least a prescribed portion of a prescribed time period, a battery life of the battery falling below a threshold value. The prescribed time period is at least 2 hours, and the prescribed portion of that time period could be at least 90%.

Methods of using this device may comprise securing the device to a subject such that the device becomes placed in the first orientation when the subject is in a first body orientation and determining the body orientation of the subject based on a reading from the first pair of electrodes. The subject may be a human, and in certain cases this human may be no more than three years old. The device can be secured to an anatomical region of the human, which may rely on the use of adhesive on the subject. The device itself may comprise this adhesive, such as on a back thereof.

After an alarm of the device sounds in response to a circuit being connected for at least a prescribed portion of a prescribed time period, the method could include a step of changing a body orientation of the subject. The device can include a communication link for transmitting a signal based on the electrode readings of the device. If necessary, this signal may cause the external device to trigger a warning, which could indicate that the subject needs attention, that the device needs to be replaced, or the device’s battery is running low.

A body-orientation monitoring system may include a wearable orientation device configured to be secured relative to a subject and configured to generate orientation data indicative of a body orientation of the subject. One or more computing devices may receive the orientation data from the wearable orientation device and may identify an orientation event of the subject based on the orientation data. The orientation event may include, for example, a detected body orientation, a change between body orientations, or a body orientation maintained for a prescribed duration.

In certain embodiments, the one or more computing devices may classify the orientation event as a clinical event type based on one or more qualification conditions. The one or more qualification conditions may include a duration threshold, an angle threshold, a circuit-completion condition, an electrode-reading condition, a protocol-defined condition, or a combination thereof. The clinical event type may include, for example, a qualifying offloading event, a non-qualifying movement, a monitored body-orientation event, a head-of-bed elevation event, a shear-risk event, a missed-repositioning event, or another clinically relevant orientation event. The one or more computing devices may generate an event record including at least the clinical event type and a time associated with the orientation event, and may generate a caregiver notification based on the event record.

In other embodiments, the wearable orientation device may include multiple pairs of electrodes associated with different orientations of the wearable orientation device. Effects of gravity may cause conductive material in an internal chamber of the wearable orientation device to complete different circuits between different pairs of electrodes when the wearable orientation device is placed in different orientations. The orientation data may include electrode readings indicating completion of one or more of the circuits. The one or more computing devices may identify the orientation event based on completion of a circuit for at least a prescribed portion of a prescribed time period, based on a change in the electrode readings indicating a change between orientations, or based on another relationship between the electrode readings and a body orientation of the subject.

Other embodiments of the present disclosure may include a method of monitoring operation of a body-orientation monitoring system. The method may include receiving system-integrity data at a computing device. The system-integrity data may be associated with monitoring availability of the body-orientation monitoring system. The system-integrity data may include, for example, battery-status data, communication-status data, attachment-status data, alignment-status data, sensor-status data, alert-output-status data, storage-status data, documentation-status data, or status-signal data. The computing device may determine a system-integrity status of the body-orientation monitoring system based on the system-integrity data. The system-integrity status may indicate that monitoring availability of the body-orientation monitoring system is functional or degraded.

In further embodiments, the computing device may generate a system-integrity alert or a monitoring-active indication based on the system-integrity status. The monitoring-active indication may indicate that body-orientation monitoring is active, available, or functional. The system-integrity alert may indicate that monitoring availability is degraded, unavailable, unreliable, or requires caregiver attention. Monitoring availability may be degraded, for example, when a battery level falls below a threshold value, a communication link becomes disconnected, one or more status signals are missed, the wearable orientation device is not properly attached, the wearable orientation device is misaligned, an alert-output pathway is unavailable, or an event record cannot be stored or transmitted through a selected documentation channel.

In yet further embodiments, the computing device may generate a position alert when the orientation data satisfies a monitored body-orientation threshold. The computing device may differentiate the system-integrity alert from the position alert by using a different sound pattern, vibration pattern, light pattern, displayed icon, displayed text, priority level, escalation rule, destination device, or other caregiver-perceptible output characteristic. The computing device may also generate a restored-monitoring indication when the system-integrity status indicates that monitoring availability has been restored.

All of these embodiments are contemplated to be within the scope of this disclosure. These and other embodiments will become readily apparent to those skilled in the art form the following detailed description of the preferred embodiments having reference to the attached figures, the disclosure not being limited to any particular preferred embodiment.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is an adult subject having a first embodiment of a device according to the present disclosure secured to them;

FIG. 2 is a child subject having a second embodiment of a device according to the present disclosure secured to them;

FIG. 3 is a front view of the first embodiment of the device of FIG. 1 according to the present disclosure;

FIG. 4 is a front cross-sectional view of the first embodiment of the device of FIG. 2 at a first orientation;

FIG. 5 is a front cross-sectional view of the first embodiment of the device of FIG. 2 at a second orientation; and

FIG. 6 is an exemplary body orientation monitoring system architecture according to the present disclosure.

Common reference numerals are used throughout the drawings and the detailed description to indicate the same elements.

### DETAILED DESCRIPTION

Systems and methods are disclosed with which a patient’s body orientation can be monitored. A device comprising a housing, one or more pairs of electrodes in an internal chamber defined by the device, and a conductive material in the internal chamber can be secured to a subject, such as a patient in a hospital bed. Due to the effects of gravity, the conductive material may, depending on the device’s orientation, push up against a pair of electrodes and complete a circuit therebetween. The device’s orientation, and which circuit is completed in that orientation, can be correlated to one or more body orientations of the subject. Electrode readings, which can come in the form of simple, binary YES/NO data can be recorded to determine the subject’s body orientation. The device can be configured to sound an alarm or trigger a warning on an external device if a threshold is reached, such as the subject being placed in a certain body orientation (e.g., facedown) or if the subject has remained in a certain body orientation for a prescribed portion of a prescribed period of time, such as 90% or more of at least a 2 hour period. Such a device may have improved battery life and usability when compared to prior body orientation measuring devices.

The present disclosure encompasses various embodiments of systems and methods for monitoring a body orientation of a subject. The subject matter disclosed herein will be best understood in view of the drawings, in which exemplary embodiments of the systems and methods disclosed herein are shown. The drawings are intended to illustrate examples and are thus not intended to limit the systems and methods they embody. The detailed description set forth below in connection with the appended drawings is intended as a description of several currently contemplated embodiments and is not intended to represent the only form in which the disclosed subject matter may be developed or utilized. This description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

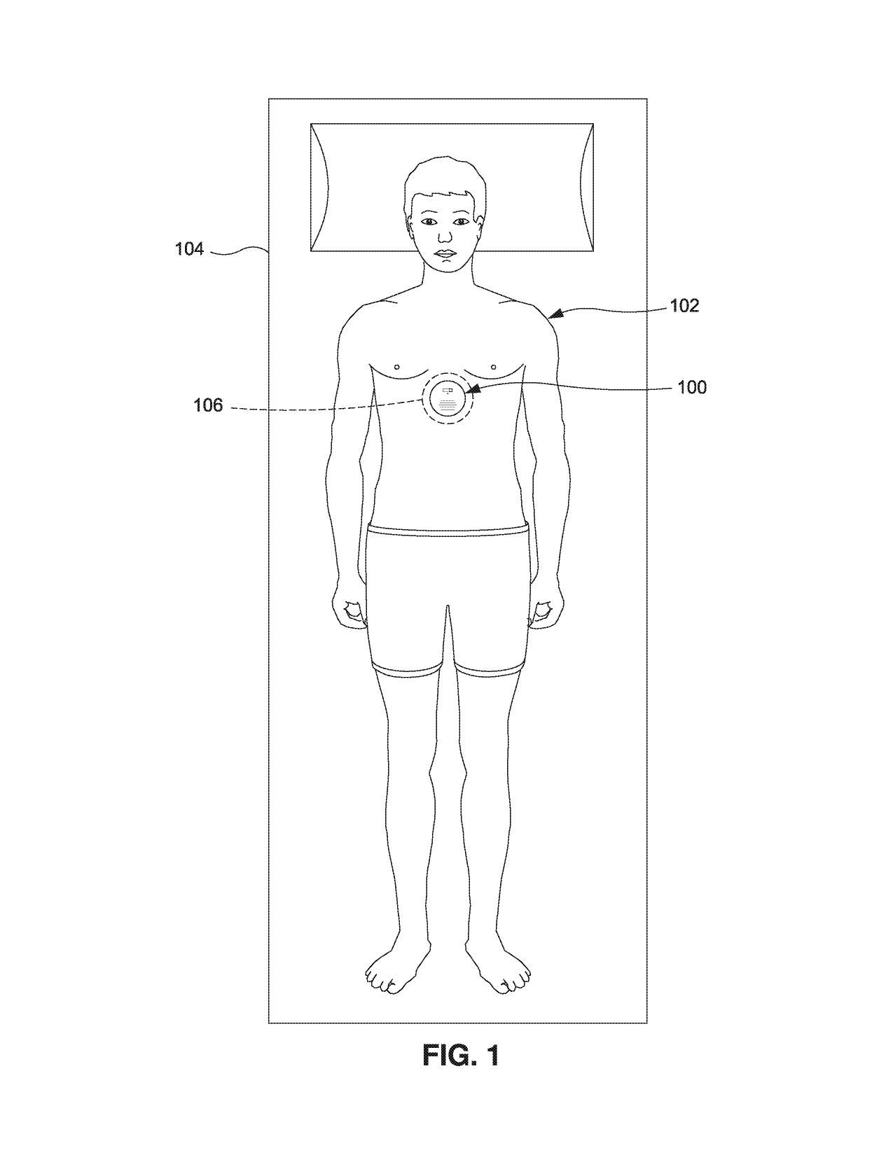

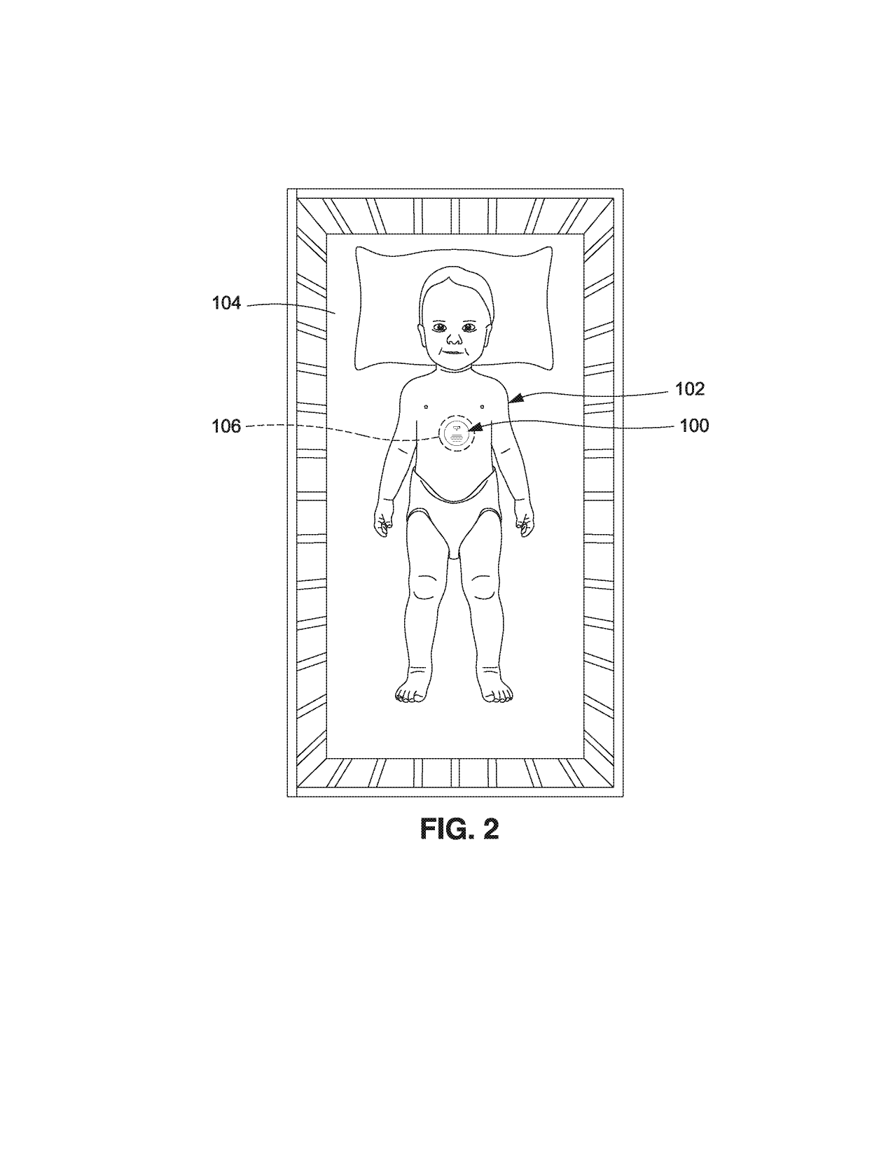

Referring first to FIGS. 1 and 2, adult and child subjects having a first embodiment of a device according to the present disclosure secured to them is shown. A device 100 may be employed to monitor the body orientation of a subject 102. The subject 102 could be resting or sleeping on a bed 104, and in some embodiments the subject 102 could be a hospital patient on a hospital bed, a baby (i.e., a human no more than three years old) in a cradle at home, or an animal such as a dog. In such contexts, the device 100 may help to prevent pressure injuries (i.e., bed sores, pressure sores, decubitus ulcers) and/or prevent the subject from laying facedown for an extended period of time (which could prevent injuries in children such as sudden infant death syndrome or SIDS). A device 100 would preferably be secured to the subject 102 such that a change in body orientation of the subject 102 causes the device 100 to have a similar change to its own orientation. This could be achieved by securing the device 100 to an anatomical region 106 like the chest or lower neck of a subject 102; to secure the device 100 in this manner, the device 100 may have a skin-friendly adhesive and/or an adhesive tape could be used to affix the device 100 to the anatomical region 106. Alternatively, a device 100 could be attached to an implement the subject 102 could be wearing, which could come in the form of a collar or tight t-shirt. Whichever option is chosen, care may be taken to ensure the orientation of the device 100 properly and appreciably changes as the body orientation of the subject 102 changes. This could depend on which body orientations one wishes the device 100 to detect and how many different body orientations a particular device 100 is capable of detecting.

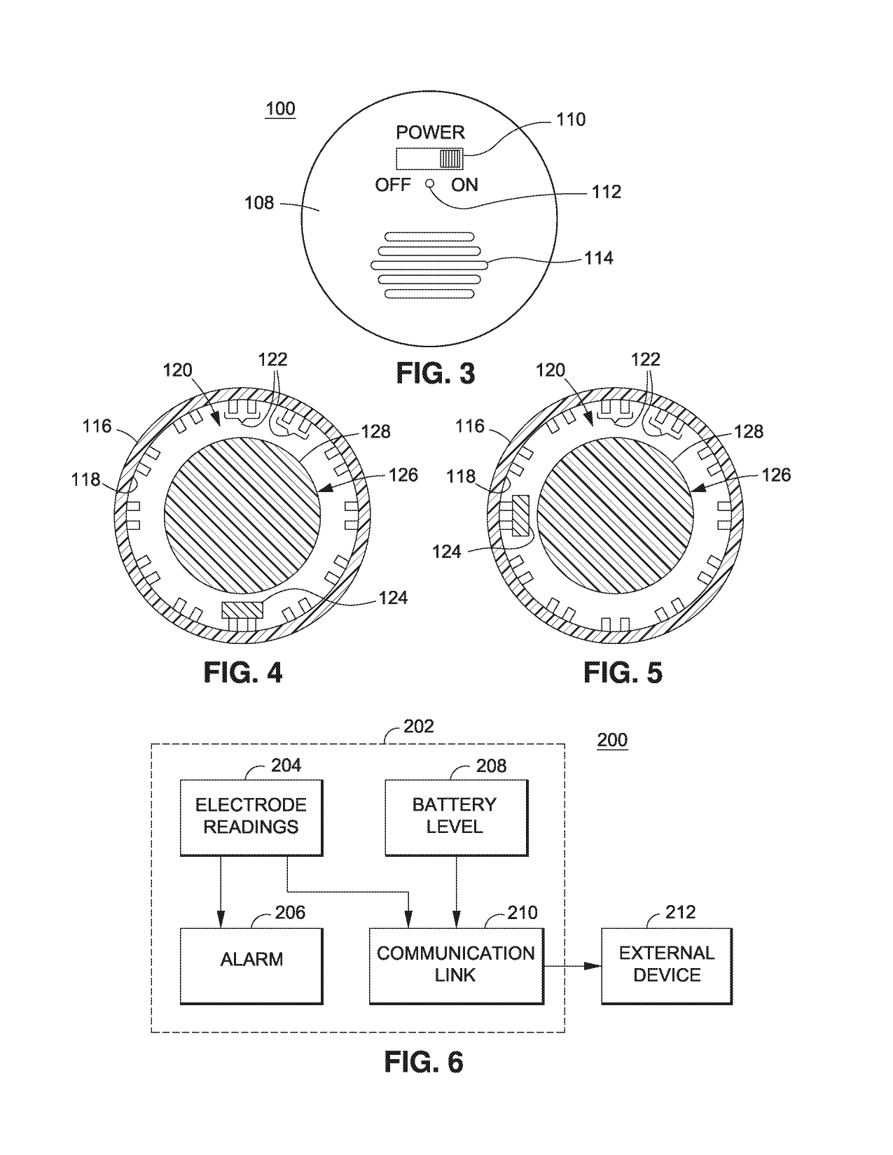

Looking now to FIG. 3, a front view of a first embodiment of a device according to the present disclosure is shown. A device may be comprised of a housing 108 which may be sized and shaped for a particular application; for example, the housing 108 could be larger or smaller if it is to be used on an adult or child respectively. Ideally, the housing 108 would have a generally smooth geometry without edges so that it may not irritate or harm a subject the device 100 is secured to. Along similar lines, the device 100 would preferably be lightweight, small, planar, and unobtrusive to reduce discomfort a subject 102 may experience with a larger device.

The housing 108 may contain a battery to power the device 100. To turn on the device 100, one may interact with a power switch 110, which could come in the form of a sliding switch, a button, etc. A power light 112 could be included to indicate if the device 100 is turned on or not. The power light 112 may remain unlit when the device 100 is powered off. When the device 100 is turned on, the power light 112 may be lit, although depending on the configuration of the device 100, the state of the power light 112 could change in response to the current battery level. For example, if the battery level is above a threshold value, the power light 112 could emit light in a first color, such as green, but when the battery level falls below that threshold value (e.g., 20%, 15%, or 10% of the maximum battery level), the power light 112 could then emit light in a second color, like red. This could indicate that one should replace the device 100 with another device 100, replace the battery in the device 100, or recharge the battery in the device 100. The power light 112 could, independently in each high-battery level and low-battery level conditions, constantly emit light or periodically emit light, such as a flash or pulse of light every 5 seconds, 10 seconds, etc. To keep power consumption low and allow a device 100 to remain activated over longer periods of time, the latter configuration is preferred. In alternative embodiments, there may be more than one power light 112, with one power light indicating whether or not the device 100 is on or off and another power light indicating that the battery level has fallen below a threshold value.

The housing 108 could define alarm vents 114 through which sounds generated by an alarm of the device 100 may be transmitted through the surrounding environment. This alarm could sound in response to the device 100 being placed in a particular undesired orientation, the device 100 remaining in a particular orientation for a prescribed portion of a prescribed period of time, and/or a battery level falling below a threshold. In this last case, and if the device 100 has a power light 112 which responds to the battery level falling below a first threshold value as described above, the alarm could sound when the battery level falls below a second, lower threshold value. In this respect, the alarm could act as a second layer of safety, as in the event that one doesn’t notice or doesn’t properly react to an indication of a low battery level from the power light 112, the alarm could sound to remind or alert them to react before the battery fully drains. The sound emitted from the alarm may be distinct for each circumstance the alarm is designed to sound for, such that one can be made aware of which condition requires their attention.

Turning now to FIGS. 4 and 5, a front cross-sectional view of the first embodiment of the device of FIG. 2 at a first orientation and a second orientation respectively are shown. A device 100 can be configured to define an internal chamber 120. The internal chamber 120 may be bounded by an internal surface 118, and this internal surface 118 could be part of the housing 108 such that an external surface 116 of the housing is associated with the external environment while the internal surface 118 is associated with the internal chamber 120 (although the internal surface 118 does not necessarily need to be part of the housing in this manner). The volume of the internal chamber 120 may be restricted by a filling 126, which could assist in limiting the movement of the conductive material 124, as will be discussed momentarily.

One or more pairs of exposed, disconnected electrodes 122 may be disposed within the internal chamber which may be activated when the device 100 is turned on. Each pair of electrodes 122 may correspond to a distinct orientation of the device 100. A conductive material 124 may be present in the internal chamber 120 which can complete a circuit between each pair of electrodes 122. The circuit which the conductive material 124 completes may depend on the orientation of the device 100 and the effect of gravity on the conductive material 124. With respect to the depiction shown in FIG. 3, for example, the conductive material 124 can connect the circuit between a first pair of electrodes 122 at the bottom of device 100 when the device is in a first orientation (e.g., when the device is secured to a subject who is laying on their back). When the device 100 then is placed in a second orientation (e.g., when the device is secured to a subject who is laying on their right-hand side), the conductive material 124 may move through the internal chamber 120 to then complete a circuit between a new pair of electrodes 122, such as the one on the left shown in FIG. 4.

The pairs of electrodes 122 can be disposed on the inner surface 118 bounding the internal chamber 120. Preferably, this inner surface 118 has a smooth geometry (e.g., circular, oval, parabolic, etc.) so that the conductive material 124 has no trouble or minimal trouble moving from one pair of electrodes 122 to the next when the orientation of the device 100 is changed; square or rectangular geometries, on the other hand, may create a risk of the conductive material 124 becoming stuck on an edge or turn and not properly completing a circuit of a particular pair of electrodes 122. In a similar vein, the pairs of electrodes 122 may be unobtrusive so that they don’t hinder the movement of the conductive material 124. The pairs of electrodes 122 shown in FIGS. 3 and 4 are intentionally made large for ease of reference, but in actual embodiments the pairs of electrodes 122 may barely protrude from the internal surface 118. Pairs of electrodes 122 may alternatively or additionally be placed on a surface 128 of the filler 126, although these pairs of electrodes 122 may need to be configured differently (e.g., protrude out further) from those disposed on the inner surface 118, as gravity may not assist in pushing the conductive material 124 against those pairs of electrodes 122. The filling 126 may intentionally limit the open volume of the internal chamber 120 so that the conductive material 124 is guided through a particular path when the orientation of the device 100 changes.

Pairs of electrodes 122 can be included or excluded in a device 100 as desired. For example, if there is only one general orientation of the device 100 at interest, only a first pair of electrodes 122 may be present. This could be the case if the subject is a baby sleeping in a crib at their home and one only wishes to be made aware when the baby lies facedown to prevent, for example, sudden infant death syndrome (SIDS). A second, third, fourth, etc. pair of electrodes 122 can be included should one desire to detect or monitor a second, third, fourth, etc. orientation of the device 100. Pairs of electrodes 122 may be spaced from one another so that they correspond to a particular orientation of the device 100 and the conductive material 124 may complete the circuit for the corresponding pair of electrodes 122 when the device 100 is in that particular orientation. The pairs of electrodes 122 may be spaced such that the device 100 needs to be rotated a certain amount in order to transition from a first orientation to a second orientation. For example, it may require at least a 30 degree, 60 degree, or 90 degree rotation on an axis of rotation for the device 100 to go from a first orientation, in which the conductive material 124 completes the circuit of a first pair of electrodes 122, to a second orientation, in which the conductive material 124 completes the circuit of a second pair of electrodes 122. The size of the conductive material 124, the spacing between pairs of electrodes 122, and the spacing between the two electrodes which make up a pair of electrodes 122 may be configured such that the conductive material 124 is only capable of being in contact with one or two electrodes of a particular pair of electrodes 122 for any given orientation of the device 100.

The conductive material 124 can be any type of conductive material familiar to those skilled in the art, including metals, electrolytes, semiconductors, nonmetallic conductors like graphite and conductive polymers, and more. The conductive material 124 could be in a solid or liquid state when exposed to an ambient temperature the device 100 will be exposed to for its intended purpose. Preferably, the conductive material 124 would be inert and maintain its conductive properties for the anticipated lifespan of the device 100 (which may be related to the battery life of a battery of the device 100).

Looking now to FIG. 6, an exemplary body orientation monitoring system architecture according to the present disclosure is shown. A body orientation monitoring system architecture 200 may include a device system architecture 202 (which may represent the functionality of the device 100). Electrode readings 204 may be received, for example, by a processor when the conductive material 124 completes a circuit for a certain pair of electrodes 122. Electrode readings 204 can take the form of a binary YES/NO measurement of each pair of electrodes 122 such that if the conductive material 124 is completing a circuit for a certain pair of electrodes 122, a YES can be recorded for that pair of electrodes and a NO can be recorded for the remaining pairs of electrodes. If there is only one/a limited range of body orientations at interest, the conductive material 124 may not complete any circuit of the pairs of electrodes 122, in which case a NO can be recorded for each pair of electrodes 122. The electrode readings 204 can be continuously monitored or recorded once over regular time intervals (e.g., an electrode reading can be taken once every 5 minutes, once every 10 minutes, and so on). From these electrode readings 204, one can determine the body orientation of a subject, which could come in the form of finding a particular body orientation of the subject or finding that the subject is not in a particular body orientation. The binary nature of the electrode readings 204 may keep power consumption of the device 100 low and allow it to be used for longer periods of time without needing to replace the device 100, replace the battery, or recharge the battery.

If the electrode readings 204 reach a threshold condition, an alarm 206 of the device 100 may be sounded. This threshold condition could be the circuit of a particular pair of electrodes 122 being complete (which could be associated with the subject being facedown), a particular pair of electrodes 122 maintaining a YES measurement over a certain period of time (e.g., 2 hours), and/or a certain percentage of the YES measurements being from a particular pair of electrodes 122 over a period of time (e.g., 90% or more of the measured readings being YES from a particular pair of electrodes over a 2-hour period). The sound emitted by the alarm for a certain threshold condition may vary from another, such that one can react to the alarm 206 accordingly. 2 hours is the threshold hospitals typically use for rotating a patient in a hospital bed to prevent pressure injuries. Therefore, this device 100 can be used to notify a hospital technician when a bedridden patient has remained in the same body orientation or mostly remained in the same body orientation over a 2-hour period, after which the technician may change the patient’s body orientation accordingly.

The device 100 could include a communication link 210 for communication with an external device 212. Ideally, the communication link 210 would comprise a wireless connection with the external device 212. The external device 212 could be a computer or mobile device which can receive the electrode readings 204. If the device 100 is being used at a hospital, the electrode readings 204 can be stored and saved under the subject’s patient file for future reference. If a threshold condition is reached for the measured electrode readings 204 (such as those which trigger the alarm 206 as described above), the external device 212 may recognize this and trigger a warning. This system could act in place of the alarm 206 (in which case the alarm 206 could be excluded from the device 100) or it can be present alongside the alarm 206 and act as an extra layer of safety. If both the alarm 206 and communication link 210 are present in a device 100, and should the communication link 210 become disconnected from the external device 212, the alarm 206 may sound indicating that such has occurred. The battery level 208 of a battery of the device 100 can also be transmitted by a communication link 210 to an external device 212 so as to inform one of when the battery will need to be replaced/recharged or the device 100 itself replaced.

The device 100 and the external device 212 may be components of a monitoring system configured to support body-orientation monitoring, event documentation, caregiver notification, institutional documentation, and system-integrity monitoring workflows. In some implementations, the device 100 may be referred to as a wearable orientation device when the device 100 is configured to be worn by, adhered to, attached to, carried by, or otherwise secured relative to the subject 102 to generate orientation data indicative of body orientation of the subject 102. The monitoring system may include the device 100, the communication link 210, the external device 212, one or more caregiver devices, one or more institutional devices, one or more bedside hubs, one or more base stations, one or more local or remote servers, one or more electronic health record interfaces, one or more printers, one or more memory devices, and one or more communication networks. The external device 212 may be implemented as, or may communicate with, a mobile phone, tablet, computer, nursing-station device, bedside receiver, caregiver wearable device, parent-room display, crib-adjacent receiver, institutional server, local server, cloud server, electronic health record system, or documentation output device. Processing and storage functions described herein may be performed by a computing device, including but not limited to, the device 100, the external device 212, a bedside hub, a base station, a server, or combinations thereof.

The monitoring system may transmit, receive, generate, process, store, display, or output information associated with operation of the device 100. Such information may include electrode readings 204, orientation data derived from the electrode readings 204, battery level 208, alert data, alarm data, communication-status data, device-status data, attachment-status data, caregiver-acknowledgment data, institutional-documentation data, event records, and system-integrity data. As used herein, an orientation event may include a detected body orientation of the subject, a transition between body orientations of the subject, or a determination that the subject has remained in a body orientation for a predetermined duration or portion of a predetermined duration. For the purposes of the present disclosure, an event record may include data generated, stored, transmitted, printed, displayed, downloaded, or otherwise output in association with an orientation event, a caregiver acknowledgment, an alarm condition, a warning condition, a communication condition, a power condition, or another operating condition of the monitoring system. Further, as used herein, a system-integrity event may include a condition indicating that monitoring, communication, power, attachment, sensing, alerting, storage, or documentation capability of the monitoring system is unavailable, degraded, or requires attention.

The monitoring system may be configured differently for different use environments while using the same general architecture. The adult-care or institutional-care environment may include a hospital, skilled nursing facility, nursing home, long-term care facility, rehabilitation facility, assisted-living facility, hospice facility, home-care environment, or another environment in which a subject 102 is monitored for body orientation, repositioning, pressure offloading, or caregiver response. For an adult-care or institutional-care environment, the external device 212 may operate with an institutional documentation system to create, store, transmit, or output records associated with patient repositioning, pressure offloading, missed repositioning intervals, caregiver acknowledgments, and device status. For an infant-care environment, the external device 212 may operate as a caregiver device, base station, or caregiver notification system to generate alerts associated with infant body orientation and to generate alerts associated with monitoring availability or system integrity. In each environment, the communication link 210 may be a wired or wireless link, and the monitoring system may use local processing, remote processing, distributed processing, local storage, remote storage, or distributed storage to carry out the functions described herein. The communication link 210 may include one or more wired or wireless communication links, including Bluetooth, Bluetooth Low Energy (BLE), Wi-Fi, cellular, ultra-wideband, near-field communication, radio-frequency communication, a wired serial connection, a network connection, or combinations thereof.

The monitoring system may be configured to generate institutional documentation associated with patient repositioning, patient body orientation, pressure offloading, missed repositioning intervals, caregiver acknowledgments, and operating status of the device 100. The institutional documentation may be generated automatically based at least partly on electrode readings 204, orientation data derived from the electrode readings 204, timing data, alarm data, communication-status data, device-status data, caregiver input, or combinations thereof. The institutional documentation may be generated at or near a time at which an orientation event, alarm condition, warning condition, caregiver acknowledgment, or system-integrity event is detected, thereby providing a contemporaneous record associated with operation of the monitoring system.

An event record generated by the monitoring system may include a time, date, timestamp, duration, event type, patient identifier, subject identifier, bed identifier, room identifier, facility identifier, device identifier, external-device identifier, caregiver identifier, orientation state, orientation transition, alarm status, warning status, acknowledgment status, escalation status, storage status, transmission status, print status, download status, or combinations thereof. The event record may indicate whether the event record was generated automatically by the monitoring system, manually acknowledged by a caregiver, manually confirmed by a caregiver, supplemented by caregiver input, or generated by a combination of automatic detection and caregiver input. The event record may also identify whether the corresponding event satisfied a prescribed repositioning interval, failed to satisfy a prescribed repositioning interval, reset an offloading timer, triggered a missed-repositioning warning, or resulted in escalation.

The monitoring system may distinguish between a detected change in body orientation and a documented repositioning or offloading event. For example, a short or transient movement may be recorded as an orientation event without being treated as a qualifying offloading event, while a lateral turn, upright posture, or other posture maintained for a predetermined duration may be recorded as a repositioning event, an offloading event, or both. The monitoring system may in turn generate institutional documentation that identifies not only that movement occurred, but also how the movement was characterized for purposes of a repositioning protocol or pressure-injury prevention protocol.

The institutional documentation may be used to support a care workflow in which caregivers are notified of pending or missed repositioning intervals and in which detected events are recorded without relying on retrospective manual charting. A caregiver may acknowledge an alarm or warning through the external device 212, another institutional device, a bedside hub, a base station, or another user interface of the monitoring system. The acknowledgment may be associated with the event record while preserving an indication that the repositioning event was detected by the device 100, acknowledged by the caregiver, confirmed by the caregiver, or otherwise supplemented by caregiver input. The monitoring system may store or output the institutional documentation for a shift, day, admission, care episode, facility-defined reporting period, or other monitoring period.

The monitoring system may output an event record or other institutional documentation through one or more documentation channels available in a care environment. The documentation channels may include an electronic health record interface, an institutional documentation system, an institutional server, a local server, a cloud server, a nursing-station receiver, a bedside hub, a bedside receiver, a local computer, a tablet, a mobile device, a network printer, a bedside printer, a nursing-station printer, memory of the device 100, memory of the external device 212, memory of a bedside hub, memory of a base station, removable memory, or another storage or output device. The monitoring system may use a single documentation channel or a plurality of documentation channels for the same event record.

The monitoring system may determine an output mode for an event record based on a facility configuration, a user configuration, network availability, electronic health record availability, printer availability, device status, communication status, power status, event type, alert priority, or combinations thereof. For example, a repositioning event, offloading event, missed-repositioning event, caregiver acknowledgment, or device-status event may be transmitted to an electronic health record system when an electronic health record interface is available. The same event record may be stored locally, transmitted to a bedside hub, transmitted to an institutional server, printed, cached for later upload, or downloaded at a later time when another documentation channel is unavailable or when redundant documentation is desired.

The monitoring system may include fallback logic for preserving institutional documentation when a main documentation channel is unavailable. The local memory may operate as an event cache or buffer that temporarily stores event records, monitoring-session data, status-signal data, documentation-status information, or combinations thereof until communication with the electronic health record system, institutional documentation system, server, external device 212, bedside hub, base station, printer, or other destination is available. If communication with an electronic health record system or institutional server is interrupted, the device 100, the external device 212, a bedside hub, a base station, or another component of the monitoring system may store the event record in local memory and associate the event record with a transmission-pending status. When communication is restored, the stored event record may be transmitted, uploaded, synchronized, or otherwise reconciled with the electronic health record system, institutional documentation system, server, or other destination. The event record may include a creation time associated with detection of the event and a later transmission time associated with output of the event record.

The monitoring system may also provide redundant documentation. For example, the system may store an event record in memory of the device 100 while transmitting the event record to the external device 212, store the event record in a bedside hub while transmitting the event record to an institutional server, transmit the event record to an electronic health record system while printing a local record, or store the event record in local memory while making the event record available for later download. Redundant documentation may be used to reduce loss of event records caused by communication interruptions, network outages, printer unavailability, server unavailability, power interruptions, or other operating conditions.

A printed record may be generated as one example of a documentation output and need not be the single documentation output or the main documentation output. The printed record may include information from one or more event records and may be generated at a bedside printer, nursing-station printer, network printer, local computer, bedside hub, external device 212, or other documentation output device. The printed record may be generated automatically upon detection of a predetermined event type, upon caregiver request, at a scheduled time, at the end of a shift, at the end of a monitoring period, or when another documentation channel is unavailable. A print status may be associated with the event record to indicate whether the event record was printed, not printed, queued for printing, failed to print, or printed after a delay.

The monitoring system may classify orientation events into one or more clinical event types based at least partly on electrode readings 204, orientation data derived from the electrode readings 204, timing data, caregiver input, facility configuration data, or combinations thereof. The clinical event type may identify how a detected orientation state or orientation transition is to be interpreted for a patient-care workflow. The classification may distinguish, for example, a left lateral turn, a right lateral turn, a return to a supine position, an upright sitting posture, an upright-to-eat posture, a head-of-bed elevation event, a prolonged non-repositioning event, a missed repositioning interval, a qualifying offloading event, a non-qualifying movement, or a shear-risk posture.

The monitoring system may classify an orientation event using one or more classification rules. A classification rule may compare orientation data to an angle threshold, a duration threshold, a sequence of orientation states, a time-of-day condition, a caregiver input, a patient-care protocol, a facility-defined protocol, or combinations thereof. For example, the monitoring system may classify an orientation event as a lateral turn when the orientation data indicates that the patient has been turned to a left-side or right-side posture and the posture is maintained for at least a predetermined duration. The monitoring system may classify an orientation event as a transient movement or a non-qualifying movement when the orientation data indicates a change in position that does not satisfy a duration threshold, angle threshold, protocol condition, or other qualification condition.

The monitoring system may separately classify orientation events that correspond to elevated or upright postures. For example, the monitoring system may classify an orientation event as a head-of-bed elevation event when the orientation data indicates that a torso portion of the patient is elevated above a predetermined angle threshold. The predetermined angle threshold may be about 30 degrees, greater than 30 degrees, less than 30 degrees, or another threshold selected by a facility or caregiver. The monitoring system may classify an orientation event as an upright sitting posture or upright-to-eat posture based on the detected orientation state, the duration of the orientation state, the time of day, caregiver input indicating a meal or activity, or combinations thereof.

Different clinical event types may be associated with different downstream actions by the monitoring system. A lateral turn may be treated as a qualifying offloading event and may reset a repositioning interval. An upright sitting posture or upright-to-eat posture may be separately recorded and may reset, conditionally reset, or not reset the repositioning interval depending on a facility-defined protocol. A head-of-bed elevation event may be logged separately from a lateral turn and may be associated with a shear-risk condition when the elevated posture is maintained for a predetermined duration or occurs repeatedly during a monitoring period. For example, a head-of-bed elevation event may be associated with a sacral shear-risk condition when the torso of the subject 102 remains elevated above a threshold angle for a predetermined duration, occurs repeatedly during a monitoring period, or occurs without a qualifying lateral offloading event. A short movement, partial movement, or movement that fails to satisfy a qualification condition may be recorded without resetting the repositioning interval.

The monitoring system may generate an event record that identifies the clinical event type assigned to an orientation event. The event record may identify the classification rule applied, the orientation state or orientation transition detected, the duration of the orientation state, whether a qualification condition was satisfied, and whether the classification caused an alarm, warning, interval reset, missed-repositioning determination, escalation, storage operation, transmission operation, print operation, or other documentation output. When more than one clinical event type applies to the same orientation event, the monitoring system may record multiple clinical event types or may apply a priority rule to select a designated clinical event type and one or more secondary clinical event types.

The monitoring system may evaluate clinical event types to determine whether a prescribed repositioning protocol has been satisfied during a monitoring period. The prescribed repositioning protocol may define a repositioning interval, an offloading requirement, one or more qualifying offloading events, one or more non-qualifying movements, one or more alert thresholds, one or more escalation thresholds, or combinations thereof. The repositioning interval may be any predetermined time interval defined by a facility, caregiver, clinician, administrator, patient-care protocol, or patient-specific care plan. As non-limiting examples, the repositioning interval may be two hours, approximately two hours, less than two hours, greater than two hours, or another predetermined interval. For example, in an institutional-care implementation, the repositioning interval may be a two-hour repositioning interval, a Q2 repositioning interval, a facility-defined turn interval, a patient-specific turn interval, or another interval specified by a care protocol. The monitoring system may start, stop, pause, resume, or reset a repositioning timer based at least partly on a clinical event type assigned to an orientation event.

The monitoring system may determine whether a qualifying offloading event occurs before expiration of the repositioning interval. A qualifying offloading event may include a left lateral turn, a right lateral turn, an upright sitting posture, an upright-to-eat posture, another posture that reduces pressure at a monitored anatomical region, or another orientation event that satisfies a facility-defined or caregiver-defined qualification condition. The qualification condition may include an angle threshold, a duration threshold, a sequence of orientation states, a minimum time away from a prior orientation state, a caregiver confirmation, a patient-care protocol condition, or combinations thereof. A non-qualifying movement may include a transient movement, a partial turn, a movement maintained for less than a threshold duration, a movement that does not sufficiently change pressure loading, or a movement that fails to satisfy another qualification condition.

The monitoring system may apply different interval-reset rules to different clinical event types. A lateral turn maintained for a predetermined duration may reset the repositioning timer. An upright sitting posture or upright-to-eat posture may reset the repositioning timer, conditionally reset the repositioning timer, or be recorded without resetting the repositioning timer according to a facility-defined protocol or patient-specific care plan. A return to a supine position may be recorded as an orientation event and may start or continue a repositioning interval for a later offloading event. A head-of-bed elevation event may be recorded separately from a lateral turn and may trigger a shear-risk determination when the elevated posture is maintained for a predetermined duration, exceeds a threshold angle, occurs repeatedly, or is combined with another risk condition. A transient movement or other non-qualifying movement may be recorded without resetting the repositioning timer.

When the monitoring system determines that no qualifying offloading event has occurred within the repositioning interval, the monitoring system may generate a missed-repositioning determination. The missed-repositioning determination may cause an alarm, warning, display message, haptic output, audible output, visual output, institutional notification, caregiver notification, event record, escalation, or combinations thereof. The monitoring system may provide an early warning before expiration of the repositioning interval, a missed-repositioning warning upon expiration of the repositioning interval, and an escalation warning when the missed-repositioning warning remains unacknowledged for a predetermined time. A caregiver acknowledgment may stop, silence, defer, escalate, or annotate the warning according to a facility configuration while preserving a record of the missed-repositioning determination and the acknowledgment.

The monitoring system may generate compliance data for a patient, bed, room, caregiver assignment, shift, day, admission, care episode, unit, facility, or other reporting period. The compliance data may include a number of completed repositioning intervals, a number of missed repositioning intervals, a percentage of completed repositioning intervals, a duration of non-compliance, cumulative time in each orientation state, cumulative time in one or more elevated or shear-risk postures, a number of alerts, a number of escalations, a caregiver acknowledgment time, a time between a warning and a qualifying offloading event, or combinations thereof. The compliance data may be included in an event record, stored by the monitoring system, displayed to a caregiver, transmitted to an institutional documentation system, printed, downloaded, or otherwise output as described herein.

The monitoring system may maintain a timeline of orientation events, clinical event types, interval-reset events, missed-repositioning determinations, caregiver acknowledgments, and escalation events. The timeline may identify why a repositioning timer was reset, why a repositioning timer was not reset, whether a warning was generated before or after expiration of the repositioning interval, and whether a caregiver response occurred within a facility-defined response period. The timeline may be used to generate a shift summary, a deviation report, an admission-level report, a patient-specific compliance report, or another report associated with a pressure-injury prevention protocol.

To support reliability, traceability, and auditability, the monitoring system may associate each event record with event-source data indicating how the event record was generated or supplemented. The monitoring system may associate each event record with event-source data indicating how the event record was generated or supplemented. The event-source data may indicate that an event was automatically detected by the device 100, automatically classified by the monitoring system, acknowledged by a caregiver, confirmed by a caregiver, edited by a caregiver, annotated by a caregiver, generated by an institutional device, generated by a bedside hub, generated by a base station, or generated by another component of the monitoring system. The event-source data may distinguish an automatically detected repositioning or offloading event from a caregiver acknowledgment of an alarm or warning, thereby preserving an indication of whether the event was detected by the monitoring system, confirmed by a caregiver, or supplemented by caregiver input.

The monitoring system may maintain an audit trail associated with an event record. The audit trail may identify creation of the event record, storage of the event record, transmission of the event record, printing of the event record, downloading of the event record, synchronization of the event record, acknowledgment of the event record, annotation of the event record, correction of the event record, or escalation associated with the event record. When an event record is corrected, annotated, supplemented, or otherwise modified after creation, the monitoring system may preserve the original event record or original event data and may store later-entered information as an additional entry, annotation, or linked record. In this manner, a later correction or caregiver note may be associated with the event record without erasing the originally generated event information.

The monitoring system may store event records in an append-only log, write-once memory, limited-edit log, audit-controlled database, local memory of the device 100, memory of the external device 212, memory of a bedside hub, memory of a base station, server memory, removable memory, or combinations thereof. The monitoring system may generate or store record-integrity data associated with one or more event records. The record-integrity data may include a checksum, hash, digital signature, sequence number, event counter, time synchronization data, device-status data, transmission-status data, print-status data, download-status data, synchronization-status data, or other data usable to verify, order, or reconcile event records.

The monitoring system may record documentation-status information for an event record. The documentation-status information may indicate whether the event record was stored locally, transmitted to an external device, transmitted to an institutional documentation system, transmitted to an electronic health record system, printed, queued for printing, downloaded, cached for later upload, synchronized after a communication interruption, delayed, failed to transmit, failed to print, or otherwise unavailable for a documentation channel. If a communication interruption, network outage, printer unavailability, server unavailability, power interruption, or other operating condition prevents output of the event record through a selected documentation channel, the monitoring system may preserve the event record and associate the event record with a pending, delayed, failed, or later-completed documentation status.

The monitoring system may use the stored event records and associated audit data to generate a traceable record of orientation events, clinical event types, repositioning-timer resets, missed-repositioning determinations, caregiver acknowledgments, escalations, communication interruptions, and documentation outputs during a monitoring period. The traceable record may identify when an event was detected, when the event was recorded, when the event was output through a documentation channel, whether caregiver action occurred, and whether later-entered information was added. The traceable record may thereby support institutional review of a pressure-injury prevention protocol while preserving distinctions between automatically generated monitoring data and later caregiver-entered information.

As described above with reference to the device 100 secured to the subject 102, the orientation of the device 100 may change with the body orientation of the subject 102 when the device 100 is secured at an anatomical region 106. The anatomical region 106 was described above as including, for example, the chest or lower neck of the subject 102. For an adult-care or institutional-care environment, the anatomical region 106 may further include a sternal region, upper chest region, lower chest region, abdominal region, lateral torso region, or another torso region from which orientation of the device 100 may be correlated with body orientation of the subject 102. Selection of the anatomical region 106 may depend on patient size, patient anatomy, care setting, adhesive tolerance, monitoring objective, facility protocol, caregiver preference, or the body orientations to be detected by the device 100.

In certain implementations of the present disclosure, the anatomical region 106 may include a forehead region of the subject 102. For example, the device 100 may be affixed to the forehead region using a skin-friendly adhesive, adhesive tape, an adhesive patch, a band, a wrap, a cap, a headband, or another attachment structure selected to maintain coupling between the device 100 and the forehead region during a monitoring period. When affixed to the forehead region, the device 100 may generate orientation data indicative of head orientation of the subject 102, and the monitoring system may use the orientation data to monitor head-positioning patterns of the subject 102. In particular, monitoring the head-positioning patterns may be useful in positional plagiocephaly applications and other infant-care applications, including by identifying prolonged maintenance of a head orientation or insufficient variation in head orientation over time.

The securement arrangements described above may be extended for adult-care or institutional-care use while preserving the requirement that the orientation of the device 100 properly and appreciably changes as the body orientation of the subject 102 changes. As described above, the device 100 may have a skin-friendly adhesive, adhesive tape may be used to affix the device 100 to the anatomical region 106, or the device 100 may be attached to an implement worn by the subject 102, such as a collar or tight t-shirt. In adult-care or institutional-care implementations, the skin-friendly adhesive may be provided on a rear surface of the housing 108, may be provided as a removable adhesive layer, or may be provided as part of a disposable or replaceable attachment component. Adhesive tape, a patch, a band, a wrap, a clip, a pocket, or another suitable attachment structure known in the art may also be used to hold the housing 108 at the selected anatomical region 106 during a monitoring period. The attachment structure may be selected to allow ordinary patient movement, bedding contact, clothing contact, patient repositioning, patient transfer, patient hygiene procedures, and caregiver handling while maintaining sufficient coupling between the device 100 and the subject 102 for body-orientation monitoring.

As described above, the housing 108 may be sized and shaped for a particular application. For instance, the housing 108 may have a generally smooth geometry with rounded edges and may be lightweight, small, planar, and unobtrusive to limit irritation or harm and reduce discomfort for the subject 102. For adult-care or institutional-care use, these housing characteristics may be implemented in a low-profile wearable form factor. In certain embodiments, the housing 108 may be generally planar, disc-like, rounded, smooth-edged, sealed, water-resistant, cleanable, disposable, reusable, rechargeable, or combinations thereof. The size, shape, thickness, edge profile, material, and surface finish of the housing 108 may be selected to reduce interference with bedding, clothing, patient movement, caregiver repositioning, patient transfer, or patient hygiene while allowing the internal chamber 120, pairs of electrodes 122, conductive material 124, and filling 126 to operate as described above.

As detailed above, the device 100 may be secured at different anatomical regions 106 and by different securement arrangements. Accordingly, the monitoring system may use calibration data or configuration data to correlate orientation of the device 100 with body orientation of the subject 102. Calibration may occur at any point during use, including but not limited to, during setup, after attachment of the device 100 to the subject 102, upon caregiver command, at a start of a monitoring period, after replacement or repositioning of the device 100, or upon detection of a potential misalignment condition. For the purposes of the present disclosure, calibration may include storing a baseline orientation, receiving caregiver input identifying the anatomical region 106 or securement arrangement, confirming a known patient posture, comparing orientation data derived from the electrode readings 204 to expected orientation data, adjusting one or more angle thresholds, adjusting one or more classification rules, or combinations thereof. The monitoring system may use the calibration data or configuration data to distinguish a supine posture, lateral posture, upright posture, head-of-bed elevation posture, or other monitored posture.

The power, indicator, and alarm features detailed above may also be configured for institutional monitoring workflows. As described above, the housing 108 may contain a battery, the power switch 110 may be used to turn on the device 100, the power light 112 may indicate whether the device 100 is turned on or may change state in response to battery level, and the alarm vents 114 may transmit sounds generated by the alarm 206. For adult-care or institutional-care use, the device 100 may include one or more additional indicators, ports, contacts, switches, buttons, or interfaces associated with power status, battery status, communication status, charging status, monitoring-active status, alarm status, warning status, memory status, attachment status, readiness status, or combinations thereof. A single indicator may provide different outputs for different operating conditions, and multiple indicators may provide separate outputs for separate operating conditions. The device 100 may also transmit corresponding status information through the communication link 210 to the external device 212, a bedside hub, a base station, an institutional device, or another component of the monitoring system.

The adult wearable configurations described in this section may use the same orientation-sensing principle described above. For example, the internal chamber 120, pairs of electrodes 122, conductive material 124, filling 126, battery, alarm 206, communication link 210, and processing of electrode readings 204 may be used to generate orientation data when the device 100 is secured at the selected anatomical region 106 of the subject 102. The monitoring system may then apply the documentation, classification, interval-reset, missed-repositioning, and record-integrity functions described herein to the orientation data generated by the device 100 without requiring a different orientation sensor or a different basic device architecture.

As described above, the subject 102 may be a human, such as a baby (i.e., a human no more than three years old) in a cradle at home, and the device 100 may be used to prevent the subject 102 from laying facedown for an extended period of time. In infant-care implementations, the device 100 may be configured to monitor whether the subject 102 assumes a prone, facedown, side, rollover, inverted, or other monitored body orientation and to cause a caregiver notification when a threshold condition associated with the monitored body orientation is satisfied. The threshold condition may include completion of a circuit by the conductive material 124 between a particular pair of electrodes 122, maintenance of that circuit for a predetermined duration, a percentage of electrode readings 204 corresponding to the monitored body orientation during a predetermined period, and/or another condition derived from the electrode readings 204.

In certain embodiments, the device 100 may be implemented as, or included in, an infant sensor pod configured to be secured to or associated with the subject 102 so that orientation of the infant sensor pod changes with body orientation of the subject 102. The infant sensor pod may include the housing 108, the internal chamber 120, one or more pairs of electrodes 122, the conductive material 124, the filling 126, a battery or other power source, the alarm 206, communication circuitry for the communication link 210, control circuitry, timing circuitry, and memory. The infant sensor pod may generate orientation data from the electrode readings 204 and may transmit the orientation data, alert data, alarm data, battery level 208, device-status data, or other operating data to the external device 212 or to another component of the monitoring system.

The infant sensor pod may be secured directly to an anatomical region 106 of the subject 102 using a skin-friendly adhesive, adhesive tape, a removable adhesive layer, an adhesive patch, or another attachment structure. The anatomical region 106 may generally include the chest, lower neck, abdominal region, torso region, diaper-adjacent region, or another body region from which orientation of the infant sensor pod may be correlated with body orientation of the subject 102. The infant sensor pod may also be associated with an implement worn by the subject 102. For infant-care implementations, the implement may include a garment, sleep sack, swaddle, onesie, shirt, diaper, diaper cover, band, wrap, pocket, pouch, clip, fabric holder, or other infant-associated support structure. Whichever securement or support arrangement is used, the arrangement may be selected to maintain sufficient coupling between the infant sensor pod and the subject 102 for body-orientation monitoring.

The external device 212 may operate as, for example, a caregiver notification device in the infant-care implementations. The caregiver notification device may include a smartphone, tablet, bedside receiver, wearable caregiver device, smart speaker, dedicated base station, crib-adjacent receiver, parent-room display, another suitable device known in the art, or combinations thereof. The caregiver notification device may receive data from the infant sensor pod through the communication link 210 and may generate a visual, audible, haptic, displayed, or transmitted notification associated with a monitored body orientation of the subject 102. The caregiver notification device may also display or store orientation data, alert history, battery level 208, device-status data, monitoring-session data, or other operating data received from the infant sensor pod.

The infant-care implementations may distinguish between a position alert associated with body orientation of the subject 102 and a system-integrity alert associated with monitoring availability. The position alert may be generated when the monitoring system determines that the subject 102 has met or exceeded a certain monitored body orientation threshold. The system-integrity alert may be generated when the monitoring system determines that the device 100, infant sensor pod, external device 212, caregiver notification device, base station, communication link 210, power source, attachment arrangement, sensing circuitry, alert output, or another component or function of the monitoring system is unavailable, degraded, improperly configured, or otherwise requires caregiver attention.

The system-integrity alert may be associated with a condition that affects the ability of the monitoring system to detect, communicate, or notify regarding a body-orientation condition. The system-integrity alert may be generated when the monitoring system determines that the device 100 or infant sensor pod is offline, out of communication range, not transmitting, low on battery, at a battery level below a low-battery threshold, not powered, malfunctioning, removed from the subject 102, displaced relative to an attachment structure, misaligned relative to the subject 102, improperly inserted into a support structure, unable to complete a self-test, unable to store data, unable to communicate with the external device 212, unable to cause a caregiver notification to be generated, or combinations thereof.

The monitoring system may determine whether a caregiver notification device or other alert-output component is capable of providing a caregiver-perceptible notification. For example, the monitoring system may determine whether notifications are enabled, a permission associated with notifications is enabled, wireless communication is enabled, an application is permitted to run in the background, audible output is available, haptic output is available, visual output is available, a caregiver notification device has adequate power, a caregiver notification device is muted or in a mode that suppresses notifications, a base station is connected, a secondary caregiver device is available, or combinations thereof. A system-integrity alert may also be generated when a caregiver notification pathway or other component used for notification delivery is unable to provide a caregiver-perceptible notification.

The monitoring system may use a status signal, check-in signal, communication-status signal, polling signal, or response signal to evaluate whether monitoring remains active and reliable. The status signal may be transmitted by the device 100 or infant sensor pod periodically, intermittently, upon request, in response to a polling signal, during setup, during a monitoring session, during a readiness check, after a detected orientation event, after a detected system-integrity event, after movement of the subject 102, after reconnection, or at another time selected by the monitoring system. Receipt of the status signal may indicate that the transmitting component is powered, operating, and able to communicate through at least a portion of the communication link 210.

The status signal may include data usable to evaluate communication status, device status, monitoring availability, or combinations thereof. For example, the status signal may include a device identifier, pod identifier, timestamp, battery level 208, signal-strength value, communication-quality value, current orientation state, last known orientation state, electrode-reading status, sensor status, attachment-status data, memory status, firmware status, error code, motion status, self-test result, monitoring-session identifier, or combinations thereof.

The monitoring system may monitor a power-source status of the device 100 or infant sensor pod as part of the system-integrity monitoring described herein. The power source may include a battery, coin-cell battery, replaceable battery, rechargeable battery, thin-film battery, capacitor, wireless-power receiver, energy-harvesting power source, or another power supply suitable for powering the device 100 or infant sensor pod during a monitoring period. The power-source status may include the battery level 208, a voltage value, a charging state, an estimated remaining operating time, a battery-presence state, a battery-installation state, a battery-compartment state, a discharge rate, a power-stability value, or combinations thereof. The monitoring system may generate a system-integrity alert, withhold a monitoring-active indication, request charging or replacement, or store an event record when the power-source status indicates that the device 100 or infant sensor pod is low on battery, at a battery level below a low-battery threshold, not powered, improperly charged, unable to finish an expected monitoring session, or otherwise unable to support reliable body-orientation monitoring.

The monitoring system may perform a readiness check or self-test before or during a monitoring session to determine whether the device 100 or infant sensor pod is ready for body-orientation monitoring. The readiness check or self-test may occur when the device 100 and/or infant sensor pod is powered on, when the infant sensor pod is inserted into an infant-associated support structure, when a caregiver begins a sleep session, at periodic intervals during operation, after a detected impact or drop, after battery replacement or charging, after reconnection to the caregiver notification device, or upon caregiver command. The readiness check or self-test may verify battery status, communication status, receipt of a recent status signal, electrode function, sensor continuity, movement of the conductive material 124, memory function, control-circuit function, attachment status, alignment status, alert-generation capability, caregiver-device communication, or combinations thereof. If the readiness check or self-test fails, the monitoring system may withhold a monitoring-active indication, generate a system-integrity alert, display a setup instruction, or notify the caregiver that monitoring is unavailable or unreliable.

The monitoring system can identify an invalid, inconsistent, or faulty orientation-sensing state as part of the readiness check, self-test, or system-integrity monitoring. For example, the monitoring system can determine that the conductive material 124 is stuck, blocked, bridged across an unanticipated combination of electrodes 122, unable to move within the internal chamber 120, providing a continuous circuit at one pair of electrodes 122 for longer than an expected duration, providing no circuit at any pair of electrodes 122 when a circuit is expected, providing simultaneous circuits at incompatible pairs of electrodes 122, producing noisy electrode readings 204, producing electrode readings 204 inconsistent with recent motion or orientation history, or otherwise producing electrode readings 204 that are invalid for determining body orientation. In response, the monitoring system can generate a system-integrity alert, withhold or downgrade a monitoring-active indication, request caregiver inspection or repositioning of the device 100 or infant sensor pod, perform a recalibration routine, store a fault record, or combinations thereof.

The monitoring system may determine that communication is degraded or unavailable based on one or more missed status signals, missed check-in signals, missed polling responses, signal-strength values, communication-quality values, packet-loss values, round-trip delays, wireless-handshake failures, repeated reconnection attempts, loss of pairing, and/or other communication-status parameters. The monitoring system may generate a weak-signal warning before complete loss of communication occurs, may generate a system-integrity alert when communication has been lost for a predetermined duration or for a predetermined number of expected status signals, and may generate a restored-communication indication when communication is restored. The system-integrity alert, weak-signal warning, or restored-communication indication may be generated by the device 100, infant sensor pod, external device 212, caregiver notification device, base station, server, or another component of the monitoring system.

The monitoring system can adjust a status-signal interval or a response to missed status signals based on an operating state of the monitoring system. For example, the monitoring system can use a shorter status-signal interval during setup, during a readiness check, after detected movement of the subject 102, after a rollover event, when orientation data is near a threshold condition, after a communication interruption, during caregiver-requested heightened monitoring, or when another condition indicates that more frequent confirmation of monitoring availability is desired. The monitoring system can use a longer status-signal interval during stable monitoring to conserve power. The monitoring system can also provide graduated responses to missed status signals, such as tolerating an initial missed status signal, generating a passive warning after a first threshold number of missed status signals or a first threshold elapsed time, generating a caregiver-perceptible system-integrity alert after a second threshold number of missed status signals or a second threshold elapsed time, and escalating the system-integrity alert if communication is not restored or if caregiver acknowledgment is not received.

The position alert and the system-integrity alert may be differentiated such that a caregiver may easily identify whether the alert relates to body orientation of the subject 102 or to monitoring availability. The alerts may differ by tone, sound pattern, volume, vibration pattern, light color, light pattern, displayed icon, displayed text, priority level, escalation rule, destination device, or combinations thereof. For example, a first alert type may indicate that the subject 102 is in a prone or facedown orientation, while a second alert type may indicate that monitoring is unavailable because communication with the infant sensor pod has been lost. The second alert type may be generated even when no prone or facedown orientation has been detected.

The monitoring system may maintain separate operating states for body-orientation status and system-integrity status. For example, the monitoring system may determine that the subject 102 is in a desired orientation while also determining that the battery level 208 is below a threshold, the communication link 210 is unreliable, or the caregiver notification device is unable to generate notifications. Conversely, the monitoring system may determine that system integrity is acceptable while also determining that a position alert is to be generated. Maintaining separate alert categories may reduce a likelihood that absence of a position alert is incorrectly interpreted as confirmation that monitoring is active and reliable.

When communication is lost, the monitoring system may preserve a last known orientation state, last known battery level 208, last received status-signal time, last known signal-strength value, last known device-status value, and/or other last known operating data and may store this last known orientation state in the memory of a device or devices within the system. The monitoring system may display the stored indication that current orientation monitoring is unavailable or that a displayed orientation state is based on stale data on the same device on which it is stored or on another device or devices within the system. The monitoring system may also store or transmit an event record identifying the system-integrity event, the time of detection, the component affected, the alert generated, a communication interruption, a restoration of communication, and any caregiver acknowledgment or corrective action associated with the event.

The monitoring system may escalate, route, acknowledge, clear, or record a system-integrity alert according to one or more alert rules. The alert rules may depend on an alert type, alert priority, duration of a system-integrity condition, number of missed status signals, battery level 208, last known orientation state, sleep-session state, caregiver acknowledgment, proximity between the device 100 or infant sensor pod and the caregiver notification device, availability of a base station, availability of a secondary caregiver device, or combinations thereof. If a caregiver does not acknowledge the system-integrity alert within a predetermined time, the monitoring system may increase a volume, change a sound pattern, repeat a notification, activate a local alarm 206, transmit the alert to another caregiver notification device, transmit the alert to a base station, or store an escalation event. When the system-integrity condition is corrected, the monitoring system may clear or downgrade the system-integrity alert, generate a restored-monitoring indication, resume a monitoring-active indication, or store an event record identifying the system-integrity condition, alert, acknowledgment, corrective action, escalation, or restoration of monitoring.

The monitoring system can route alerts, indications, or monitoring-session data to one or more caregiver notification devices according to caregiver configuration, caregiver role, schedule, proximity, acknowledgement status, or monitoring-session state. For example, the monitoring system can designate an initial caregiver device and one or more secondary caregiver devices, can transmit an alert simultaneously to multiple caregiver notification devices, can transmit an alert sequentially from an initial caregiver device to a secondary caregiver device if the alert is not acknowledged, can route alerts according to a day schedule, night schedule, care-shift schedule, or caregiver handoff, or can route alerts to a caregiver notification device determined to be closer to the subject 102, the device 100, the infant sensor pod, or a base station. The monitoring system can store or update caregiver-routing data so that a monitoring-session record identifies which caregiver notification device received an alert, whether the alert was acknowledged, whether the alert was escalated, and whether caregiver responsibility was transferred during the monitoring session.

The infant sensor pod may be removably coupled to an infant-associated support structure while maintaining the orientation relationship described above between the device 100 and the subject 102. The infant-associated support structure may include an adhesive patch, fabric cradle, garment cradle, sleep sack, swaddle, onesie, shirt, diaper, diaper cover, band, wrap, pocket, pouch, clip, pod holder, and/or another support structure associated with the subject 102. The infant-associated support structure may hold the infant sensor pod at a predetermined or caregiver-selected location relative to the subject 102 so that a detected orientation of the infant sensor pod may be correlated with a body orientation of the subject 102. The infant-associated support structure may be disposable, washable, reusable, replaceable, adjustable, or configured for single-use attachment to the subject 102 or to an article worn by the subject 102.

The infant-associated support structure may include one or more alignment features configured to orient the infant sensor pod in a predetermined direction relative to the body of the subject 102. The alignment features may include asymmetric pod geometry, a keyed insertion structure, a magnetic alignment feature, a snap-fit feature, a one-way insertion pocket, printed orientation markings, fabric seam alignment, an electrical contact pattern, a mechanical lockout, a shaped recess, a tab, a notch, a label, a color-coded indicator, or combinations thereof. The alignment features may reduce a likelihood that the infant sensor pod is inserted backwards, upside down, rotated relative to an intended orientation, incompletely seated, or otherwise positioned in a manner that causes orientation data derived from the electrode readings 204 to be misinterpreted.

The monitoring system may determine whether the infant sensor pod is coupled to, seated in, or aligned with the infant-associated support structure. The determination may be based at least partly on electrical continuity contacts between the infant sensor pod and the infant-associated support structure, magnetic coupling sensors, Hall-effect sensors, mechanical switches, pressure sensors, capacitive sensors, light sensors, garment-closure sensors, motion-pattern analysis, absence of expected infant micro-movements, temperature change, proximity to the subject 102, radio-frequency identification, near-field communication, pairing data associated with the infant-associated support structure, impedance detection, calibration data, orientation data, caregiver input, or combinations thereof. The monitoring system may use the determination to generate attachment-status data or alignment-status data.

The attachment-status data or alignment-status data may indicate that the infant sensor pod is attached, detached, partially detached, inserted, not inserted, incompletely seated, inverted, rotated, misaligned, removed from the subject 102, removed from the infant-associated support structure, displaced relative to the infant-associated support structure, or otherwise not positioned for reliable body-orientation monitoring. The monitoring system may generate a system-integrity alert when the attachment-status data or alignment-status data indicates that the infant sensor pod may not be sufficiently coupled to the subject 102 or may not be positioned in a manner that permits reliable correlation between orientation of the infant sensor pod and body orientation of the subject 102. The system-integrity alert may be generated before, during, or after a monitoring session and may be output by the infant sensor pod, the external device 212, a caregiver notification device, a base station, a server, or another component of the monitoring system.

The monitoring system may perform an attachment or alignment check during setup, when the infant sensor pod is powered on, when the infant sensor pod is inserted into the infant-associated support structure, when a caregiver begins a sleep session, at periodic intervals during operation, after detected movement of the subject 102, after a detected impact or drop, after replacement or repositioning of the infant sensor pod, after reconnection to the caregiver notification device, upon caregiver command, and/or at another time selected by the monitoring system. The attachment or alignment check may include confirming continuity of one or more contacts, confirming a magnetic or mechanical coupling state, comparing a detected orientation to an expected orientation, confirming that the infant sensor pod is within a predetermined angular range, confirming that expected motion or temperature characteristics are present, or prompting a caregiver to confirm placement of the infant sensor pod.

If the monitoring system determines that the infant sensor pod is improperly attached, improperly seated, misaligned, or otherwise not positioned for reliable body-orientation monitoring, the monitoring system may withhold a monitoring-active indication, display a setup instruction, generate a check-monitor alert, request caregiver repositioning of the infant sensor pod, request confirmation of the attachment location, apply a calibration correction, adjust one or more orientation thresholds, adjust one or more classification rules, store an event record, or combinations thereof. If the monitoring system determines that the infant sensor pod has been properly attached or realigned, the monitoring system may generate a readiness indication, clear or downgrade a system-integrity alert, begin or resume a monitoring session, store attachment-status data, or store alignment-status data.

The attachment and alignment features described in this section may be implemented with the device 100 described above. For example, when the device 100 is used as an infant sensor pod, the internal chamber 120, pairs of electrodes 122, conductive material 124, filling 126, and electrode readings 204 may provide orientation data as described above, and the attachment and alignment features may provide information indicating whether the device 100 is properly coupled or aligned for reliable correlation between orientation of the device 100 and body orientation of the subject 102. In other embodiments, one or more of the attachment-status, seating-status, or alignment-status features described herein may be used with another body-orientation sensing device configured to monitor orientation of the subject 102.

The monitoring system may be configured to reduce a likelihood of fail-silent operation during an infant-care monitoring session. For the purposes of the present disclosure, fail-silent operation may include a condition in which the subject 102 is not being reliably monitored, or in which an alert generated by the monitoring system may not be perceived by a caregiver, while the caregiver is not informed that monitoring availability is unavailable or degraded. To reduce a likelihood of fail-silent operation, the monitoring system may provide a monitoring-active indication only after confirming one or more system-integrity conditions, such as communication with the device 100 or infant sensor pod, receipt of a recent status signal, sufficient battery level 208, acceptable attachment status, acceptable alignment status, successful readiness check or self-test, and availability of at least one caregiver-perceptible notification pathway. Accordingly, the monitoring system may avoid treating an absence of a position alert as confirmation that monitoring is active when one or more system-integrity conditions cannot be verified.

The monitoring-active indication may be visually, audibly, haptically, textually, or otherwise output by the external device 212, caregiver notification device, base station, infant sensor pod, or another component of the monitoring system. The monitoring-active indication may identify that monitoring is active, unavailable, degraded, based on stale data, or requires caregiver action before reliable monitoring may begin or resume. The monitoring system may withhold, remove, change, or downgrade the monitoring-active indication when a system-integrity event indicates that monitoring availability is degraded.

The monitoring system may require caregiver acknowledgment before treating a monitoring-unavailable indication, failed readiness check, offline condition, pod-removal condition, misalignment condition, caregiver-notification-pathway condition, or other fail-silent condition as resolved. When the condition is corrected, the monitoring system may generate a restored-monitoring indication, resume the monitoring-active indication, store a restoration event, or require a new readiness check before indicating that monitoring is active.

In implementations of the present disclosure, the monitoring system may store or output monitoring-session data associated with a sleep session, care session, repositioning interval, institutional monitoring period, infant monitoring period, or other monitoring period. The monitoring-session data may include position alerts, system-integrity alerts, repositioning events, monitoring-active indications, monitoring-unavailable indications, status signals, missed status signals, last known orientation states, battery level 208, attachment-status data, alignment-status data, caregiver acknowledgments, alert escalations, corrective actions, restored-monitoring indications, start times, end times, or combinations thereof. For instance, in an infant-care implementation, the monitoring-session data may allow a caregiver to review whether monitoring was active, unavailable, degraded, acknowledged, corrected, or restored during a sleep session or other infant monitoring period. The monitoring-session data may be stored by the device 100, infant sensor pod, external device 212, caregiver notification device, base station, server, or combinations thereof and may be displayed, downloaded, transmitted, or otherwise output for caregiver review, institutional review, care documentation, or monitoring-system troubleshooting.

The above description is given by way of example, and not limitation. Given the above disclosure, one skilled in the art could devise variations that are within the scope and spirit of this disclosure. Further, the various features of the embodiments disclosed herein can be used alone, or in varying combinations with each other and are not intended to be limited to the specific combination described herein. Thus, the scope of the claims is not to be limited by the illustrated embodiments. Additional modifications and improvements of the present disclosure may also be apparent to those of ordinary skill in the art. Thus, the particular combination of parts and steps described and illustrated herein is intended to represent only certain embodiments of the present subject matter and is not intended to serve as limitations of alternative devices and methods within the spirit and scope of this disclosure.

What is claimed is:

1. A monitoring system for monitoring repositioning of a subject, the monitoring system comprising:
a wearable orientation device configured to be secured to the subject and configured to generate orientation data indicative of a body orientation of the subject; and
one or more computing devices configured to receive the orientation data from the wearable orientation device, the one or more computing devices comprising:
at least one computer readable memory storing software instructions; and
at least one processor coupled to the at least one computer readable memory;
wherein the at least one processor performs operations upon execution of the software instructions, the operations comprising:
identifying an orientation event of the subject based on the orientation data;
assigning a clinical event type to the orientation event based on one or more qualification conditions;
generating an event record comprising at least the clinical event type and a time associated with the orientation event; and
generating a caregiver notification based on the event record.

2. The monitoring system of claim 1, wherein the wearable orientation device comprises:
a housing defining an internal chamber;
a conductive material positioned within the internal chamber;
a first pair of electrodes positioned within the internal chamber; and
a second pair of electrodes positioned within the internal chamber.

3. The monitoring system of claim 2, wherein the first pair of electrodes is associated with a first orientation of the wearable orientation device and the second pair of electrodes is associated with a second orientation of the wearable orientation device.

4. The monitoring system of claim 3, wherein effects of gravity cause the conductive material to complete a first circuit between the first pair of electrodes when the wearable orientation device is oriented in the first orientation and to complete a second circuit between the second pair of electrodes when the wearable orientation device is oriented in the second orientation.

5. The monitoring system of claim 4, wherein the orientation data comprises electrode readings indicating completion of the first circuit and completion of the second circuit.

6. The monitoring system of claim 5, wherein identifying the orientation event comprises detecting completion of the first circuit for at least a prescribed portion of a prescribed time period.

7. The monitoring system of claim 5, wherein identifying the orientation event comprises detecting a change in the electrode readings indicating a change between the first orientation and the second orientation.

8. The monitoring system of claim 1, wherein the one or more qualification conditions comprise a duration threshold or an angle threshold.

9. The monitoring system of claim 1, wherein the clinical event type comprises a qualifying offloading event when the orientation event satisfies the one or more qualification conditions.

10. The monitoring system of claim 1, wherein the clinical event type comprises a non-qualifying movement when the orientation event fails to satisfy the one or more qualification conditions.

11. A method of monitoring operation of a body-orientation monitoring system, the method comprising:
receiving system-integrity data at a computing device, the system-integrity data being associated with monitoring availability of the body-orientation monitoring system, the body-orientation monitoring system including a wearable orientation device configured to be secured relative to a subject and configured to generate orientation data indicative of a body orientation of the subject;
determining a system-integrity status of the body-orientation monitoring system via the computing device, the system-integrity status being based on the system-integrity data; and
generating a system-integrity alert or a monitoring-active indication via the computing device based on the determined system-integrity status.

12. The method of claim 11, wherein the system-integrity data comprises a battery level of a battery of the wearable orientation device.

13. The method of claim 12, wherein determining the system-integrity status comprises determining that monitoring availability is degraded when the battery level falls below a threshold value.

14. The method of claim 13, wherein generating the system-integrity alert comprises sounding an alarm of the wearable orientation device.

15. The method of claim 11, wherein the system-integrity data comprises communication-status data associated with a communication link between the wearable orientation device and the computing device.

16. The method of claim 15, wherein determining the system-integrity status comprises determining that monitoring availability is degraded when the communication link becomes disconnected or when one or more status signals are missed.

17. The method of claim 11, further comprising receiving the orientation data at the computing device and generating a position alert when the orientation data satisfies a monitored body-orientation threshold.

18. The method of claim 17, wherein the system-integrity alert is differentiated from the position alert.

19. The method of claim 18, wherein differentiating the system-integrity alert from the position alert comprises using a different sound pattern, vibration pattern, light pattern, displayed icon, displayed text, priority level, escalation rule, or destination device.

20. The method of claim 11, further comprising generating a restored-monitoring indication when the system-integrity status indicates that monitoring availability has been restored.
